(19) United States
(12) Patent Application Publication
(10) Pub. No.: US 2024/0335680 A1
(43) Pub. Date: Oct. 10, 2024

(54) COMPOSITIONS AND METHODS RELATED TO BLOOD-BRAIN BARRIER PENETRATION

(71) Applicants: INSIGHTEC LTD., Tirat Carmel (IL); SUNNYBROOK RESEARCH INSTITUTE, Toronto (CA)

(72) Inventors: Achal Singh ACHROL, Miami, FL (US); Nir LIPSMAN, Toronto (CA); Ying MENG, Toronto (CA); Nadir ALIKACEM, Miami, FL (US)

(21) Appl. No.: 18/681,401

(22) PCT Filed: Aug. 2, 2022
(86) PCT No.: PCT/IB2022/000444

§ 371 Date: Feb. 5, 2024

Related U.S. Application Data
(60) Provisional application No. 63/229,865, filed on Aug. 5, 2021.

(51) Int. Cl. A61N 7/00; A61M 37/00

(52) U.S. Cl. CPC A61N 7/00; A61M 37/0092; A61N 2007/0026; A61N 2007/0039; A61N 2007/0065

(57) ABSTRACT

The disclosure is directed to methods related to the delivery of antibodies targeting Her2 across the blood-brain barrier.

### CROSS-REFERENCE TO RELATED APPLICATIONS

This application claims priority to U.S. Provisional Application No. 63/229,865, filed on Aug. 5, 2021, the entire contents of which are incorporated herein.

### DESCRIPTION OF THE TEXT FILE SUBMITTED ELECTRONICALLY

The content of the text file submitted electronically herewith is incorporated herein by reference in their entirety: A computer readable format copy of the Sequence Listing (Filename: “COMPOSITIONS AND METHODS RELATED TO BLOOD-BRAIN BARRIER PENETRATION.xml”; Date recorded: Jul. 25, 2022; File size: 4,096 bytes in size).

### BACKGROUND

Failure of promising therapies to cross the blood-brain barrier (“BBB”) is an important cause of poor clinical response for a broad range of brain pathologies including tumors. Even with tumor-related BBB disruption, significant morphologic heterogeneity within the brain-tumor barrier (“BTB”) often results in poor penetration of pharmaceuticals, including antibody-based targeted therapies that are typically around 150 kDa in molecular weight. The ability to safely enhance therapeutic delivery of these antibodies to pathologic tissue in a non-invasive and reproducible manner has yet to be achieved. Thus, there remains a significant need for improved compositions and methods that enable the delivery of therapeutic antibodies across the BBB.

### SUMMARY

Accordingly, in various aspects, the present disclosure relates to a method of treating a Her2+ metastatic breast tumor in the brain, comprising: (a) selecting a human patient having a Her2+ metastatic breast tumor in the brain or a recent resection of a Her2+ metastatic breast tumor in the brain; and (b) applying an ultrasound beam, e.g. a focused, magnetic resonance-guided ultrasound beam (MRgFUS), to the cranium of the human patient to cause transient disruption of the blood-brain barrier (BBB) of the selected human patient, where the selected human patient is receiving: (i) one or more antibodies targeting Her2, during, and/or after the application of the ultrasound beam, e.g. MRgFUS, and (ii) one or more microbubble compositions immediately before and/or during the application of the ultrasound beam, e.g. MRgFUS.

In other aspects, the present disclosure relates to a method of treating a Her2+ metastatic breast tumor in the brain, comprising applying to a population of patients, characterized by having Her2+ metastatic breast tumor in the brain or a recent resection of a Her2+ metastatic breast tumor in the brain, an ultrasound beam, e.g. a MRgFUS, to the cranium to cause transient disruption of the blood-brain barrier (BBB) the patients are receiving: (i) one or more antibodies targeting Her2, during, and/or after the application of the ultrasound beam, e.g. MRgFUS, and (ii) one or more microbubble compositions immediately before and/or during the application of the ultrasound beam, e.g. MRgFUS.

In embodiments, the antibody targeting Her2 is a full length antibody or an antibody format, e.g. selected from pertuzumab, trastuzumab, and ado-trastuzumab, e.g. administered by systemic infusion.

In embodiments, the Her2 antibody is labeled by adding an imaging tracer such as ¹¹¹In for in vivo detection and monitoring of therapeutic delivery and biodistribution of the anti-Her2 antibody on SPECT.

In embodiments, the microbubble compositions comprise one or more lipid-based microspheres, e.g. perflutren lipid microspheres.

In embodiments, the transient disruption of the BBB allows for movement of the antibody targeting Her2 across the BBB.

In embodiments, the tumors do not substantially increase in size or reduce in size after completion of the method. In embodiments, the tumors present as necrotic after completion of the method.

In embodiments, volumetric voxel-based analyses is used to confirm and quantify the amount of ¹¹¹In-labeled Her2 antibody that has been delivered to the target tissue, and to confirm avoidance of exposure of tissue outside of the targeted area.

In aspects, there is provided a method of in vivo detection and monitoring of therapeutic delivery and biodistribution of a therapeutic agent, e.g. a therapeutic antibody, administered in the context of application of an ultrasound beam, e.g. a MRgFUS, to the cranium of a human patient to cause transient disruption of the blood-brain barrier (BBB), the method comprising detection of the therapeutic agent, labeled by adding an imaging tracer such as ¹¹¹In.

In embodiments, volumetric voxel-based analyses is used to confirm and quantify the amount of ¹¹¹In-labeled therapeutic agent, e.g. a therapeutic antibody, that has been delivered to the target tissue, and to confirm avoidance of exposure of tissue outside of the targeted area.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

FIG. 1 shows images of magnetic resonance imaging (“MRI”) and single photon emission computed tomography (SPECT). The images across the top of FIG. 1 show contrast-enhanced T1-weighted MRI, which demonstrated the lesion in subject one (left, solid arrow, upper left image). Ultrasound was delivered to the contrast enhancing regions of the tumor, as well as to adjacent non-enhancing tissue. Color map of acoustic emissions detected during sonication, and the resultant increased enhancement in the sonication volume are shown (upper middle images, dashed arrows), indicating opening of the blood brain barrier (“BBB”). Contrast-enhanced T1-weighted MRI one day after treatment demonstrates restoration of BBB/brain-tumor barrier (“BTB”) integrity. The images across the bottom of FIG. 1 show magnetic resonance guided focused ultrasound (“MRgFUS”) using SPECT imaging. The standardized uptake value ratios (“SUVR”) of trastuzumab in the sonication volume increased by 86% from 1.18 to 2.20 for early (4 hours) imaging, and the SUVR increased by 135% from 1.84 to 4.32 for late (48 hours) imaging. Greater SUVR in delayed vs early SPECT imaging is expected given progressive accumulation of the tracer in the tumor, and is also indicative of Her2 positivity.

FIG. 2 shows images of SPECT subtraction maps, which illustrate significantly enhanced trastuzumab penetration and binding in the sonicated regions (white dotted line). The color bar in the images indicates the acoustic dose. The color map of the right column indicates the percentage change in SUVR in trastuzumab with MRgFUS relative to baseline. Changes in SUVR correspond to the sonication volume and acoustic emissions detected during sonications. Histograms (far right column) show the distribution of SUVR changes across voxels in individual lesions and average of lesions.

FIG. 3 are MRI images showing changes in an enhancing lesion following combined MRgFUS and trastuzumab treatments. Yellow (or white) circles highlight the lesion of interest for comparison.

FIG. 4 is an image showing an intraoperative photograph of a subject undergoing MRgFUS treatment for BBB opening. Picture obtained with consent of the subject.

FIG. 5 is an MRI image showing a region of interest (“ROI”) drawn on the primary motor cortex used for normalization, volume=23800 mm³.

FIG. 6 shows contrast enhanced T1-weighted MR images of lesions targeted by MRgFUS.

FIG. 7 shows MRI images showing T2* MRI prior (left), and after (right), of MRgFUS induced BBB opening in examples without (top) and with (bottom) sonication spot related T2* signal change (top). The yellow (or white) circles in the image highlight the sonicated region.

FIG. 8 shows images of SPECT subtraction colormaps for all treated lesions. These images show the percentage change in SUVR in trastuzumab with MRgFUS relative to baseline (color bar).

FIG. 9 shows SPECT images showing an unsonicated lesion in subject two demonstrating no SUVR change in the delayed ¹¹¹In labelled trastuzumab SPECT imaging. The color bar represents percentage change in SUVR. The pie graph in the upper right image shows the percentage of voxels in tumor volume with greater than 20% increase in SUVR.

FIG. 10 shows MRI images showing serial contrast enhanced T1-weighted MRI in subject two, demonstrating a progressive increase in tumor size and necrotic core during treatment followed by size reduction.

### DETAILED DESCRIPTION

The present disclosure is based, in part, on the surprising discovery of a non-invasive, spatially-targeted improvement in monoclonal antibody drug delivery across the BBB. As disclosed herein, ultrasound beam, e.g. MRgFUS, treatments significantly enhance therapeutic delivery and binding of trastuzumab to targeted tumors. In addition, the present disclosure shows how MR-guided focused ultrasound safely and reversibly increases BBB permeability within metastatic Her2-positive brain tumors and surrounding brain regions.

The present disclosure is based, in part, on the surprising discovery of an in vivo detection and monitoring method for therapeutic delivery and biodistribution of a therapeutic agent, e.g. a therapeutic antibody, administered in the context of an ultrasound beam, e.g. a MRgFUS.

### Methods of Treatment

In aspects, the present disclosure relates to a method of treating a Her2+ metastatic breast tumor in the brain, comprising: (a) selecting a human patient having a Her2+ metastatic breast tumor in the brain or a recent resection of a Her2+ metastatic breast tumor in the brain; and (b) applying an ultrasound beam, e.g. MRgFUS, to the cranium of the human patient to cause transient disruption of the blood-brain barrier (BBB) of the selected human patient, where the selected human patient is receiving: (i) one or more antibodies targeting Her2, during, and/or after the application of the ultrasound beam, e.g. MRgFUS, and (ii) one or more microbubble compositions immediately before and/or during the application of the ultrasound beam, e.g. MRgFUS.

In other aspects, the present disclosure relates to a method of treating a Her2+ metastatic breast tumor in the brain, comprising applying to a population of patients, characterized by having Her2+ metastatic breast tumor in the brain or a recent resection of a Her2+ metastatic breast tumor in the brain, an ultrasound beam, e.g. MRgFUS, to the cranium to cause transient disruption of the blood-brain barrier (BBB) the patients are receiving: (i) one or more antibodies targeting Her2, during, and/or after the application of the ultrasound beam, e.g. MRgFUS, and (ii) one or more microbubble compositions immediately before and/or during the application of the ultrasound beam, e.g. MRgFUS.

In embodiments, the present methods allow delivery of one or more antibodies targeting Her2 across the BBB, without the need for surgery. In embodiments, the present methods allow delivery of one or more antibodies targeting Her2 across the BBB, without the need for intrathecal delivery. In embodiments, the present methods allow delivery of one or more antibodies targeting Her2 across the BBB, without the need for conjugation of the one or more antibodies targeting Her2, e.g. a receptor-mediated transport (RMT) system, and the like.

### Ultrasound Beam/Application Thereof

In embodiments, the application of the ultrasound beam, e.g. MRgFUS targets at least one region or site of the brain. In embodiments, the application of the ultrasound beam, e.g. MRgFUS targets at least two regions or sites of the brain. In embodiments, the application of the ultrasound beam, e.g. MRgFUS targets at least three regions or sites of the brain. In embodiments, the application of the ultrasound beam, e.g. MRgFUS targets at least one, or two, or three regions or sites of the brain contemporaneously.

In embodiments, the treatment method is repeated at least two, or three, or four, or five, or ten times.

In embodiments, the ultrasound beam is or comprises a focused ultrasound beam. In embodiments, the ultrasound beam is or comprises a guided ultrasound beam. In embodiments, the ultrasound beam is or comprises a focused and guided ultrasound beam. In embodiments, the ultrasound beam is or comprises a magnetic resonance-guided ultrasound beam (MRgFUS). In embodiments, an ultrasound beam is or comprises a focused, computerized tomography (CT)-guided ultrasound beam. In embodiments, an ultrasound beam is or comprises a focused, positron emission tomography (PET)-guided ultrasound beam. In embodiments, an ultrasound beam is or comprises a focused, stereotactically-navigated guided ultrasound beam based on registration to prior scan.

In embodiments, the focused ultrasound, e.g. MRgFUS beam is applied directly to the human patient's cranium, e.g. using a helmet-shaped ultrasound transducer. In embodiments, the ultrasound beam, e.g. MRgFUS, is applied at a center frequency of about 180 to about 230 kHz, e.g. about 220 KHz.

In embodiments, the treatment duration is at least about 60 minutes, or at least about 90 minutes, or at least about 120 minutes, or at least about 150 minutes, or at least about 180 minutes. In embodiments, the treatment duration is about 100 minutes, or about 110 minutes, or about 120 minutes, or about 130 minutes, or about 140 minutes, or about 150 minutes, or about 160 minutes.

In embodiments, the focused ultrasound beam, e.g. MRgFUS, is applied for at least about 10 seconds, or at least about 20 seconds, or at least about 30 seconds, or at least about 40 seconds, or at least about 50 seconds, or at least about 60 seconds. In embodiments, the focused ultrasound beam, e.g. MRgFUS, is applied for about 10 seconds, or about 20 seconds, or about 30 seconds, or about 40 seconds, or about 50 seconds, or about 60 seconds.

In embodiments, the focused ultrasound beam, e.g. MRgFUS, is applied in pulses.

In embodiments, the focused ultrasound beam, e.g. MRgFUS, is applied at a power of at least about 5 W, or at least about 10 W, or at least about 15 W, or at least about 20 W, or at least about 25 W. In embodiments, the focused ultrasound beam, e.g. MRgFUS, is applied at a power of about 10 W, or about 15 W, or about 20 W.

In embodiments, the focused ultrasound beam, e.g. MRgFUS, targets and/or the tumor is present in or has been resected from one or more of the frontal lobe, parietal lobe, temporal lobe, occipital lobe, and cerebellum. In embodiments, the ultrasound beam, e.g. MRgFUS, targets and/or the tumor is present in or has been resected from the supratentorial region or site of the brain. In embodiments, the focused ultrasound beam, e.g. MRgFUS, targets and/or the tumor is present in or has been resected from the infratentorial region or site of the brain. In embodiments, the focused ultrasound beam, e.g. MRgFUS, targets and/or the tumor is present in or has been resected from the insula. In embodiments, the focused ultrasound beam, e.g. MRgFUS, targets and/or the tumor is present in or has been resected from the brainstem. In embodiments, the focused ultrasound beam, e.g. MRgFUS, targets and/or the tumor is present in or has been resected from the pons. In embodiments, the focused ultrasound beam, e.g. MRgFUS, targets and/or the tumor is present in or has been resected from the posterior fossa. In embodiments, the focused ultrasound beam, e.g.

MRgFUS, targets the and/or the tumor is present in or has been resected from corticomedullary gray/white junction. In embodiments, the focused ultrasound beam, e.g. MRgFUS, targets the and/or the tumor is present in or has been resected from the meninges.

### Patients and Patient Populations

In embodiments, the selected human patient or patient population is afflicted with Her2+ metastatic breast tumor in the brain. In embodiments, the selected human patient or patient population is afflicted with Her2+ metastatic breast tumor in the brain and has had a recent resection. In embodiments, the selected human patient or patient population is afflicted with Her2+ metastatic breast tumor in the brain and is undergoing treatment as described herein, optionally in combination with an anti-tumor combination agent, as described herein.

In embodiments, the selected human patient or patient population presents with a plurality of lesions.

In embodiments, the selected human patient or patient population presents with a plurality of lesions in at least two regions or sites of the brain, selected from, without limitation, the frontal lobe, parietal lobe, temporal lobe, occipital lobe, cerebellum, supratentorial region, infratentorial region, insula, brainstem, pons, posterior fossa, corticomedullary gray/white junction, and/or meninges.

In embodiments, the selected human patient or patient population has had recent resection of the tumor and presents with a plurality of post-resection cavities.

In embodiments, the selected human patient or patient population has had recent resection the tumor and presents with a plurality of post-resection cavities in at least two regions or sites of the brain, selected from, without limitation, the frontal lobe, parietal lobe, temporal lobe, occipital lobe, cerebellum, supratentorial region, infratentorial region, insula, brainstem, pons, posterior fossa, corticomedullary gray/white junction, and/or meninges.

In embodiments, the selected human patient or patient population is defined by the inclusion and/or exclusion criteria of Table 7.

In embodiments, the selected human patient or patient population has metastatic Her2+ breast cancer with brain metastases, that are clearly defined on pre-therapy contrast enhanced MRI. In embodiments, the selected human patient or patient population have Her2+ breast cancer with brain metastases that has progressed based on imaging

In embodiments, the selected human patient or patient population have not received a systemic anti-tumor treatment in about 1, or about 2, or about 3 weeks before the present method.

In embodiments, the selected human patient or patient population have not had a intracranial hemorrhage within at least the last 2 weeks

In embodiments, the selected human patient or patient population do not have a skull feature that can impair or reduce the ultrasound beam, e.g. calcification scars or lesions, clips or other metallic implanted objects in the skull or brain, in the ultrasound beam, e.g. MRgFUS, beam path

### Outcomes of the Method

In embodiments, the transient disruption of the BBB allows for movement of the antibody targeting Her2 across the BBB. In embodiments, this movement of the antibody targeting Her2 occurs during or for a period shortly after the transient disruption of the BBB. In embodiments, the methods described herein provide for substantially all of the disrupted BBB closing after the application of the ultrasound beam, e.g. MRgFUS. Accordingly, in embodiments, after the completion of the methods, the patient(s) have a restored, or pre-treatment, BBB. In embodiments, the disruption of the BBB is reversible.

In embodiments, the human patient or population of patients demonstrates an increased standard uptake value ratio (SUVr) of greater than about 50%, or greater than about 60%, or greater than about 70%, or greater than about 80%, or greater than about 90%, as compared to administration of antibody targeting Her2, e.g. labeled with Indium-111 in the absence of application of the focused ultrasound beam, e.g. MRgFUS, to the cranium.

In embodiments, the human patient or population of patients demonstrates an increased standard uptake value ratio (SUVr) of about 50%, or about 60%, or about 70%, or about 80%, or about 90%, as compared to administration of the antibody targeting Her2, e.g. labeled with Indium-111 in the absence of application of the focused ultrasound beam, e.g. MRgFUS, to the cranium.

In embodiments, the present method results in the present tumors not substantially increasing in size after completion of the method. In embodiments, the present method results in the present tumors reducing in size after completion of the method. In embodiments, the present method results in the present tumors being characterized as necrotic after completion of the method.

### Therapeutic Agent

In embodiments, the antibody targeting Her2 is a full length antibody or an antibody format.

In embodiments, the antibody format is selected from a single-chain antibody (scFv), a microprotein (cysteine knot protein, knottin), a DARPin; a Tetranectin; an Affibody; a Transbody; an Anticalin; an AdNectin; an Affilin; a Microbody; a plastic antibody; a single-domain antibody, a recombinant heavy-chain-only antibody (VHH), a shark heavy-chain-only antibody (VNAR), a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; an Affimer, a DuoBody, a Fv, a Fab, a Fab′, and a F(ab′)₂.

In embodiments, the antibody targeting Her2 is selected from pertuzumab, trastuzumab, and ado-trastuzumab. In embodiments, the antibody targeting Her2 is trastuzumab. In embodiments, the trastuzumab is administered at a dose of about 6 mg/kg to about 8 mg/kg.

In embodiments, the antibody targeting Her2 is administered at least about 90 minutes, or at least about 120 minutes, or at least about 150 minutes, or at least about 180 minutes, or at least about 210 minutes before the application of the ultrasound beam, e.g. MRgFUS.

In embodiments, the antibody targeting Her2 is administered about 60 minutes, or about 90 minutes, or about 120 minutes before the application of the ultrasound beam, e.g. MRgFUS.

In embodiments, the antibody targeting Her2 is administered during the application of the ultrasound beam, e.g. MRgFUS. In embodiments, the antibody targeting Her2 is administered after the application of the ultrasound beam, e.g. MRgFUS.

In embodiments, the antibody targeting Her2 is administered by systemic injection, bolus injection or slow diffusion injection.

In embodiments, the antibody targeting Her2 is administered by systemic infusion.

### Microbubbles

In embodiments, the microbubble compositions comprise one or more lipid-based microspheres. In embodiments, the microbubble compositions are perflutren lipid microspheres.

In embodiments, the microbubble compositions are administered to the patient no more than 60, or 30, or 20, or 10 minutes before the application of the ultrasound beam, e.g. MRgFUS. In embodiments, the microbubble compositions are administered to the patient throughout the method.

In embodiments, the microbubble compositions are administered by systemic injection, bolus injection or slow diffusion injection.

In embodiments, the microbubble compositions are administered by systemic infusion.

### Anti-Tumor Combination Agents

The present disclosure, in embodiments, also provides for combination therapies. In embodiments, the treatment further comprises administering an antibody-based anti-tumor combination agent. In embodiments, the antibody-based anti-tumor combination agent is a full length antibody or an antibody format. In embodiments, the antibody format is selected from a single-chain antibody (scFv), a microprotein (cysteine knot protein, knottin), a DARPin; a Tetranectin; an Affibody; a Transbody; an Anticalin; an AdNectin; an Affilin; a Microbody; a plastic antibody; a single-domain antibody, a recombinant heavy-chain-only antibody (VHH), a shark heavy-chain-only antibody (VNAR), a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; an Affimer, a DuoBody, a Fv, a Fab, a Fab′, and a F(ab′)₂. In embodiments, the antibody-based anti-tumor combination agent is a monoclonal antibody. In embodiments, the antibody-based combination agent is a bispecific antibody. In embodiments, the antibody-based anti-tumor combination agent is an antibody-drug conjugate.

In embodiments, the antibody-based anti-tumor combination agent is directed to an antigen expressed on a tumor cell or an immune cell. In embodiments, the antibody-based anti-tumor combination agent is directed to one of: CD20, optionally selected from ibritumomab tiuxetan, obinutuzumab, ofatumumab, and rituximab; CD30, optionally brentuximab; CD52, optionally alemtuzumab; EGFR, optionally selected from cetuximab, panitumumab, and necitumumab; VEGF and VEGFR2, optionally selected from bevacizumab and ramucirumab; programmed cell death protein 1 (PD-1), optionally selected from nivolumab, cemiplimab and pembrolizumab; programmed cell death ligand 1 (PD-L1), optionally selected from atezolizumab, avelumab, and durvalumab; CTLA-4, optionally ipilimumab; and CD38, optionally daratumumab.

In embodiments, the tumor express the antigen against which the antibody-based anti-tumor combination agent is directed.

### Methods of Tracking Antibodies Targeting Her2

In embodiments, the one or more antibodies targeting Her2 are labeled with a tracer label, to permit tracking of the transit of the treatment agent, wherein the tracer label optionally comprises indium-111.

In embodiments, the therapeutic delivery to the target brain regions via the non-invasive imaging tracer signal is quantified to determine the effect of improved therapeutic delivery across the BBB specific to the treatment agent being delivered

In embodiments, the effect of ultrasound beam, e.g. MRgFUS, BBB treatment on trastuzumab therapeutic delivery is measured by the imaging tracer (e.g. Indium-111) signal e.g. on SPECT/CT images co-registered to clinical contrast-enhanced T1-weighted MRI to measure standardized uptake values (SUV) within the target tumor regions as measured within each tumor voxel.

In embodiments, the SUVs are normalized to an appropriate control region's mean SUV (e.g. motor cortex or other normal brain) in order to calculate the standardized uptake value ratios (SUVRs) of each voxel across the entire tumor volume.

In embodiments, two-dimensional and/or three-dimensional volumetric heatmaps, or other similar representation, of the voxel-by-voxel percentage change in SUVR are generated using the formula: (post-MRgFUS SUVR−baseline SUVR)/(baseline SUVR) to characterize the effect of MRgFUS BBB treatment on improving the therapeutic delivery across the entire target region of the tumor volume.

### Methods of Tracking

In aspects, there is provided a method of in vivo detection and monitoring of therapeutic delivery and biodistribution of a therapeutic agent, e.g. a therapeutic antibody, administered in the context of application of an ultrasound beam, e.g. MRgFUS, to the cranium of a human patient to cause transient disruption of the blood-brain barrier (BBB), the method comprising detection of the therapeutic agent, labeled by adding an imaging tracer such as ¹¹¹In.

In embodiments, volumetric voxel-based analyses is used to confirm and quantify the amount of ¹¹¹In-labeled antibody-based treatment agent, that has been delivered to the target tissue, and to confirm avoidance of exposure of tissue outside of the targeted area.

In embodiments, the voxel-based method relates to monitoring of an antibody-based treatment agent, such therapeutic antibody including the one or more antibodies targeting Her2 and/or the anti-tumor combination agents, as described herein.

In aspects, there is provided a method for tracking therapeutic delivery and/or of an antibody-based treatment agent, comprising selecting a human patient having a tumor in the brain or a recent resection of a tumor in the brain; and applying an ultrasound beam, e.g. MRgFUS, to the cranium of the human patient to cause transient disruption of the BBB of the selected human patient; wherein, the selected human patient is receiving one or more imaging tracer-labeled antibody-based treatment agents during and/or after the application of the ultrasound beam, e.g. MRgFUS, to allow tracking of the transit of the treatment agent, and one or more microbubble compositions immediately before and/or during the application of the ultrasound beam, e.g. MRgFUS.

In embodiments, the method detects antibody-based treatment agent biodistribution in the body and/or across the BBB to a target brain region.

In embodiments, the tracer is non-invasive.

In embodiments, the tracer is or comprises a radiolabel, optionally selected from one or more of indium-111 (¹¹¹In), fluorine-18 (¹⁸F), and carbon-11 (¹¹C).

In embodiments, the therapeutic delivery to target brain regions via imaging tracer signal is quantified to determine an effect of therapeutic delivery across the BBB specific to the antibody-based treatment agent being delivered

In embodiments, the effect of ultrasound beam, e.g. MRgFUS, treatment on the antibody-based treatment agent delivery is measured by the imaging tracer signal on, e.g. SPECT/CT images co-registered to clinical contrast-enhanced T1-weighted MRI to measure standardized uptake values (SUV) within the target tumor regions as measured within each tumor voxel.

In embodiments, the SUVs are normalized to an appropriate control region's mean SUV to calculate the standardized uptake value ratios (SUVRs) of each voxel across the entire tumor volume.

In embodiments, the control region is selected from a motor cortex or other normal brain region.

In embodiments, two-dimensional and/or three-dimensional volumetric heatmaps, or other similar representation, of the voxel-by-voxel percentage change in SUVR are generated using the formula:

(post-MRgFUS SUVR−baseline SUVR)/(baseline SUVR)

to characterize the effect of MRgFUS BBB treatment on improving the therapeutic delivery across the entire target region of the tumor volume.

In embodiments, the human patient demonstrates an increased standard uptake value ratio (SUVr) of greater than about 50%, or greater than about 60%, or greater than about 70%, or greater than about 80%, or greater than about 90%, as compared to administration of antibody-based treatment agent in the absence of application of the focused ultrasound beam, e.g. MRgFUS, to the cranium.

In embodiments, the human patient demonstrates an increased standard uptake value ratio (SUVr) of greater than about 50%, or greater than about 60%, or greater than about 70%, or greater than about 80%, or greater than about 90%, as compared to administration of the antibody-based treatment agent in the absence of application of the focused ultrasound beam, e.g. MRgFUS, to the cranium.

In embodiments, the tumor in the brain is metastatic.

In embodiments, the focused ultrasound beam, e.g. MRgFUS, is applied directly to the human patient's cranium using a helmet-shaped ultrasound transducer.

In embodiments, the focused ultrasound beam, e.g. MRgFUS, is applied at a center frequency of about 220 KHz.

In embodiments, the microbubble compositions comprise one or more lipid-based microspheres, as described elsewhere herein.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

In the following examples, fifteen treatments were carried out in three subjects without any grade three or higher serious adverse events. Two mild adverse events were recorded. Successful BBB opening was achieved in all patients and was associated with a mean 87% increase in SUVR (p=0.001) on ¹¹¹In-labelled trastuzumab SPECT imaging. Voxel analysis showed SUVR increased by up to 450%, with on average 87% (range 49-99%) of sonication volume voxels seeing a greater than 20% increase in SUVR, compared to 8% (SD 8%, range 2-14%) in control lesions. MRgFUS-enhanced trastuzumab delivery was further associated with response or stability of the tumor volumes with on average 21% (SD 10%) reduction in unidimensional measurements.

### Example 1: MR-Guided Focused Ultrasound Procedure

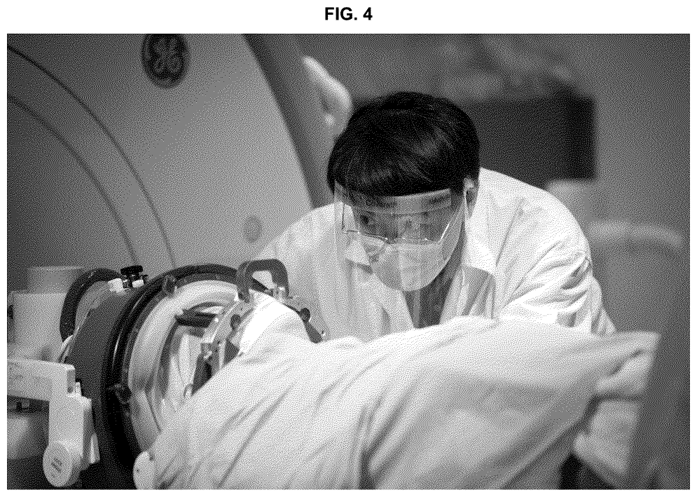

In the experiments of this example, a BBB opening was performed using a hemispheric clinical prototype for MRgFUS (FIG. 4). Patient preparations (e.g., frame placement) and general procedures were undertaken, and therapeutic infusions were coordinated with pre-procedure preparations to finish shortly before sonications. The treatment volumes, contoured to the tumor and tumor margins based on intraoperative MRIs, were prescribed by the study neurosurgeon in collaboration with the oncologist and physicist. Pulsed ultrasound was delivered and monitored in conjunction with an intravenous infusion microbubble ultrasound contrast agent (DEFINITY (Perflutren Lipid Microsphere), Lantheus). Acoustic monitoring of microbubble activity during sonications enabled ultrasound to be delivered in an automated, near-continuous manner to achieve an average cavitation dose found to produce safe BBB permeability. Follow-up MRIs were acquired within an hour, and at approximately 18-24 hours after sonications, to assess for radiographic adverse events (“AEs”), and changes in BBB/BTB permeability by expert neuroradiologist.

### Example 2: ¹¹¹In-BzDTPA-NLS-Trastuzumab Production

In the experiments of this example, clinical grade Indium-111 (¹¹¹In)-BzDTPA-NLS-trastuzumab was produced under good manufacturing practices (GMP). Briefly, reconstituted trastuzumab ((Herceptin), Roche) was conjugated with isothiocyanate ester (2-(4-isothiocyanatobenzyl)-diethylenetriaminepentaacetic acid) ((BzDTPA) Macrocyclics, Plano, TX) and nuclear localizing sequence peptides (NLS: CGYGPKKKRKVGG (SEQ ID NO: 1), Biobasic), to produce 1.0 ml aliquots of 5.0 mg of BzDTPA-trastuzumab-NLS, stored at 2-4° C. as kits. To prepare the radiopharmaceutical on the day of the administration, 3.0-4.5 mCi (111-166 MBq) of ¹¹¹In chloride ((BWXT), Ottawa, Canada) was added into a single kit vial, and incubated at room temperature for 1.5-2 hours. The radiochemical purity of ¹¹¹In-BzDTPA-NLS-trastuzumab was >98%, and ¹¹¹In-BzDTPA-NLS-trastuzumab bound with high affinity to HER2-positive SK-BR-3 human breast cancer cells (Kₐ=4.2×10⁸ L/mol and Bₘₐₓ=5×10⁵ receptors/cell).

### Example 3: SPECT Imaging Acquisition and Analysis

Gamma emission from ¹¹¹In allows in vivo detection of the tracer on single-photon emission computed tomography (“SPECT”) imaging, which can be used for measuring drug biodistribution. Each study consisted of one ¹¹¹In-BzDTPA-NLS-trastuzumab injection followed by an early (4 hours) and delayed (48 hours) SPECT scan. The injection was slowly administered intravenously after 50 mg of diphenhydramine premedication. Each participant was monitored for three hours for any infusion-related anaphylactic reactions or AEs.

In the experiments of this example, two studies were conducted in every participant, once at baseline and once during the first MRgFUS treatment, at least one week apart. When performed one week apart, an additional SPECT scan was acquired prior to the second tracer injection to confirm minimal residual radioactivity. Using medium energy collimation, SPECT acquisitions were carried out on the GE Optima 640 SPECT/CT scanner (GE Healthcare, Waukesha, WI) over 17 minutes, with 60 views in a 180° arc and 30 seconds per view. The SPECT data were reconstructed with HybridRecon-Oncology SUV SPECT (version 1.3, HERMES Medical Solutions AB, Stockholm, Sweden).

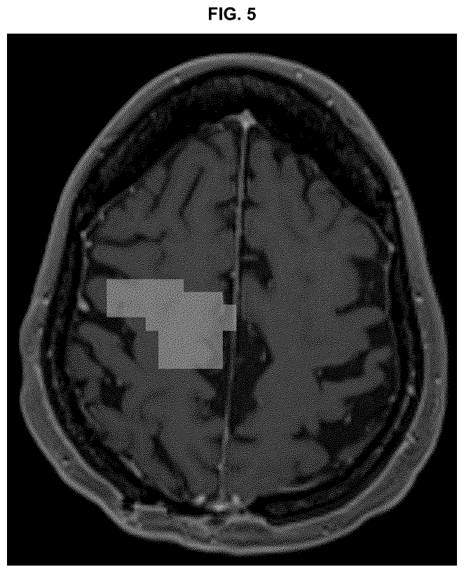

To spatially characterize the effect of MRgFUS on trastuzumab brain penetration, the SPECT/CT images were co-registered to a clinical isometric contrast enhanced T1-weighted MRI with Advanced Normalization Tools (ANTs). The mean SUVs were measured within sonication volumes, which overlapped the metastatic lesions. The treatment volume mean SUVs were then normalized to the appropriate motor cortex mean SUV (FIG. 5) to calculate the standardized uptake value ratios (SUVRs). The motor cortex mean SUVs were checked for stability between baseline and post FUS (Table 1, shown below). Finally, volumetric maps of the voxel-by-voxel percentage change in SUVR were generated using the following formula: (post-MRgFUS SUVR-baseline SUVR)/(baseline SUVR).

TABLE 1
Primary motor cortex SUVs mean and standard
deviation across SPECT studies

| | Early 4 hrs SPECT imaging | | Late 48 hrs SPECT imaging | |
|---|---|---|---|---|
| Subject | Pre FUS | Post FUS | Pre FUS | Post FUS |
| 1 | 5985 (1606) | 5863 (1484) | 5973 (2025) | 5653 (2077) |
| 2 | 3981 (1239) | 3791 (1275) | 4353 (2148) | 4296 (1825) |
| 3 | 4037 (855) | 3464 (769) | 4718 (682) | 3153 (1253) |

### Example 4: Patient Demographics

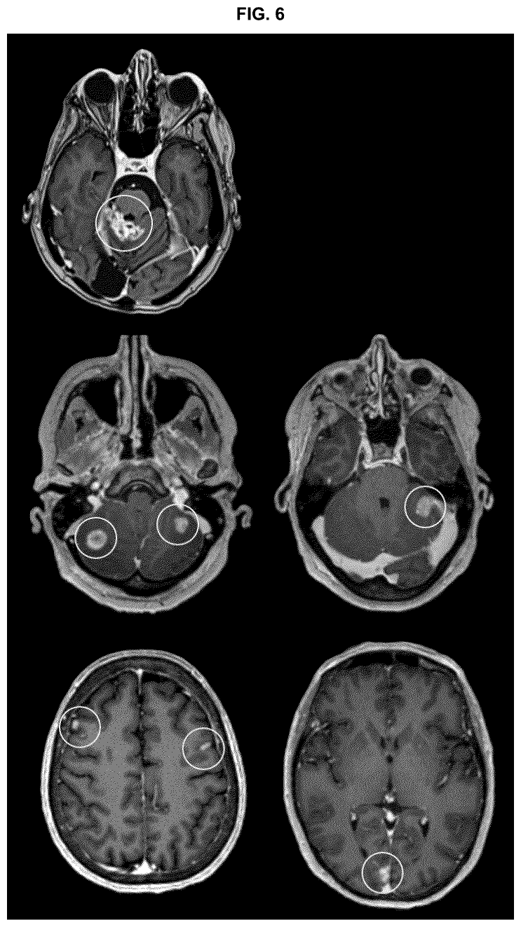

In the experiments of this example, three subjects with pathology confirmed primary Her2-positive breast cancer isolated progression of intracranial disease were enrolled in this study as described in Table 2, below. Patients were referred by an oncologist expert in the diagnosis and management of breast cancer, and approached after a multidisciplinary discussion about their suitability for the trial. The radiographic appearances of their tumors are illustrated in FIG. 6. All subjects had progressing or active intracranial disease on recent serial neuroimaging with otherwise well-controlled systemic disease. For those with a history of radiation therapy, all lesions 10 were at least six weeks from last radiation treatment. In particular, subject one and subject three received stereotactic radiosurgery four years and one year prior to the first MRgFUS treatment, respectively.

TABLE 2
Patient demographics

| | | | Pathology | | | | | |
|---|---|---|---|---|---|---|---|---|
| | | | of | Breast | Brain | | | |
| | | | primary | cancer | tumor | Lesion | Prior | Delivered |
| Subject | Age | Sex | tumor | diagnosis | diagnosis | location | Therapies | therapeutic |
| 1 | 41 | F | HER2 | 2013 | 2014 | Single | Whole | Trastuzumab, |
| | | | positive, | | | infratentorial | brain | pertuzumab |
| | | | ER/PR | | | lesion | radiation, | |
| | | | positive | | | | stereotactic | |
| | | | | | | | radiosurgery, | |
| | | | | | | | surgery | |
| 2 | 53 | F | HER2 | 2015 | 2019 | Three | Whole | Trastuzumab, |
| | | | positive, | | | infratentorial | brain | pertuzumab, |
| | | | ER/PR | | | lesions | radiation | paclitaxel |
| | | | positive | | | | | |
| 3 | 56 | F | HER2 | 2011 | 2019 | Three | Stereotactic | Trastuzumab, |
| | | | positive, | | | supratentorial | radiosurgery | pertuzumab |
| | | | ER/PR | | | lesions | | |
| | | | positive | | | | | |

### Example 5: MRgFUS BBB/BTB Disruption

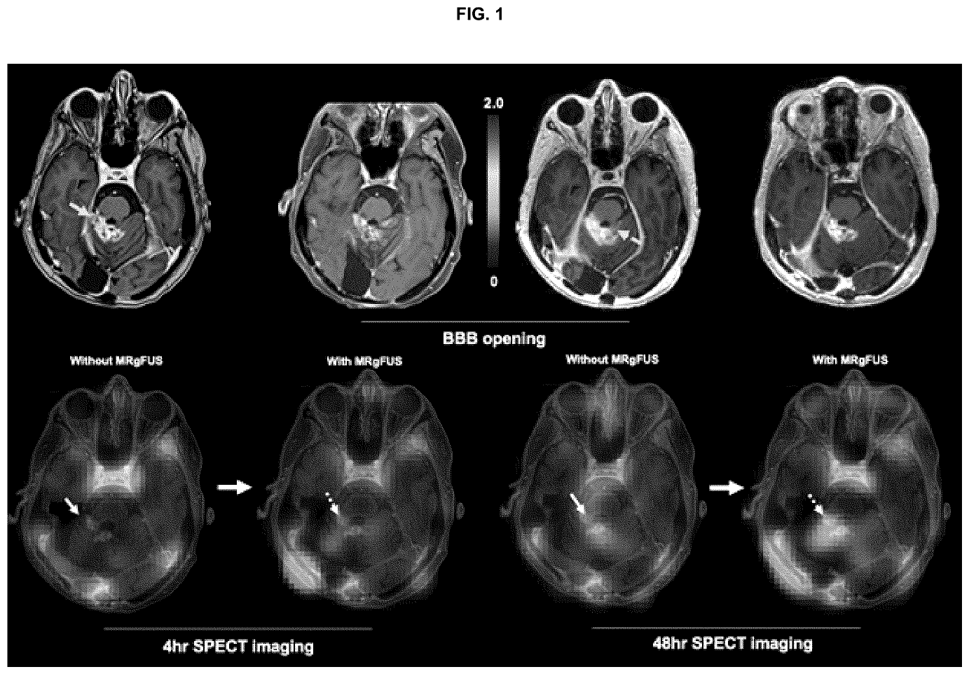

The first two subjects underwent six treatments, while the third subject underwent three treatments, amounting to fifteen total treatments. After transcranial sonication, an increased level of contrast enhancement on T1-weighted MRI was seen in all cases in both tumor and tumor margins, matching the areas of dose contour, which indicated increased permeability of the BBB (FIG. 1). The mean treatment time was 128 (SD 31) minutes to treat 24 (SD 12) cm³ mean volume, 15 (SD 5) W ultrasound power, with values per subject in Table 3, below. The duration of multi-lesion treatments improved overtime, indicating improved efficiency.

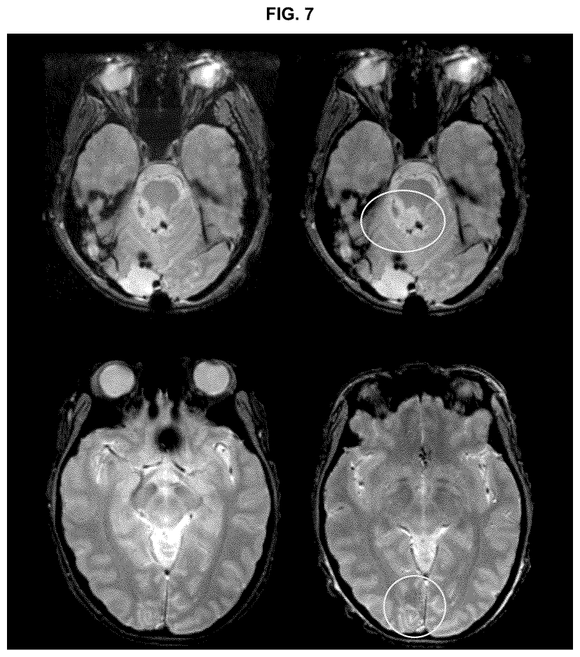

Overall, the procedures were well-tolerated, and there were no serious adverse events (AEs). Two transient and mild AEs were reported by patients, and related to placement of the headframe and MRI table (Table 4, below). Subjects were discharged within 1-2 hours following treatment. Neurologic and general physical exams were unchanged. Further neuroimaging did not reveal clinically relevant adverse events such as hemorrhage or edema. Hypointense spots on T2*-weighted MRI was visualized in [˜30%] cases, and consistent with previous reports, were asymptomatic and resolving with time (FIG. 7).

TABLE 3
Sonication parameters, SD in brackets

| | Sonication Time | | Sonication Volume |
|---|---|---|---|
| Subject | (minutes) | Power (W) | (cm³) |
| 1 | 114 (13) | 19 (4) | 16 (3) |
| 2 | 154 (31) | 14 (3) | 30 (16) |
| 3 | 106 (26) | 8 (1) | 27 (3) |

TABLE 4
Summary of treatment related adverse events

| Adverse event | N (total 15) | Severity | Duration |
|---|---|---|---|
| Pin-site | 1 | Mild | Transient |
| tenderness | | | |
| Back | 1 | Mild | Transient |
| discomfort | | | |

### Example 6: 111 In-BzDTPA-NLS-Trastuzumab Imaging

In the experiments of this example, the effect of MRgFUS on the brain, and the tumor penetration of trastuzumab was examined. In these experiments, ¹¹¹In-BzDTPA-NLS-trastuzumab was used as a radiotracer. The γ-emission from ¹¹¹In allows trastuzumab to be visualized on SPECT. On baseline SPECT/CT scans, there was minimal early (4 hours) uptake of the ¹¹¹In-BzDTPA-NLS-trastuzumab in the tumor, with some progressive uptake at the delayed (48 hours) scan, as expected due to tumor-related BBB disruption (FIG. 1). Early and delayed uptake in normal brain was minimal. These baseline studies confirmed Her2 positivity of the lesion, in agreement with histopathology by immunohistochemistry and fluorescence in situ hybridization of the patient's neurosurgical specimen.

Following MRgFUS with concurrent injection of the ¹¹¹In-BzDTPA-NLS-trastuzumab, the experiments of this example were able to directly visualize increased early and delayed signal on SPECT imaging within the sonication volume for all lesions treated (FIG. 1). To better quantify changes over the whole spatial region, the SUV ratio (SUVR) of an ROI drawn over the sonicated area normalized to the motor cortex was compared, which had stable signal across all studies (Table 1, above). The SUVR increased from 1.31 (SD 0.35) to 2.04 (SD 0.73, p=0.007, paired-t test) on the early imaging, and from 2.35 (SD 1.22) to 4.40 (SD 1.06, p=0.001, paired-t test, Table 5, below) on the delayed imaging. The latter indicated greater retention and enhanced binding of trastuzumab to Her2 receptors where ultrasound was delivered, notably in both the contrast enhancing tumor and adjacent non-enhancing tissue regions on MRI. Per lesion, the delayed SUVR increased by as much as 187%. In comparison, early and late SUVRs in two unsonicated lesions changed from 2.35 (SD 0.87) to 2.44 (SD 0.41) and 3.79 (SD 0.78) to 3.00 (SD 0.35) respectively (Table 6, below).

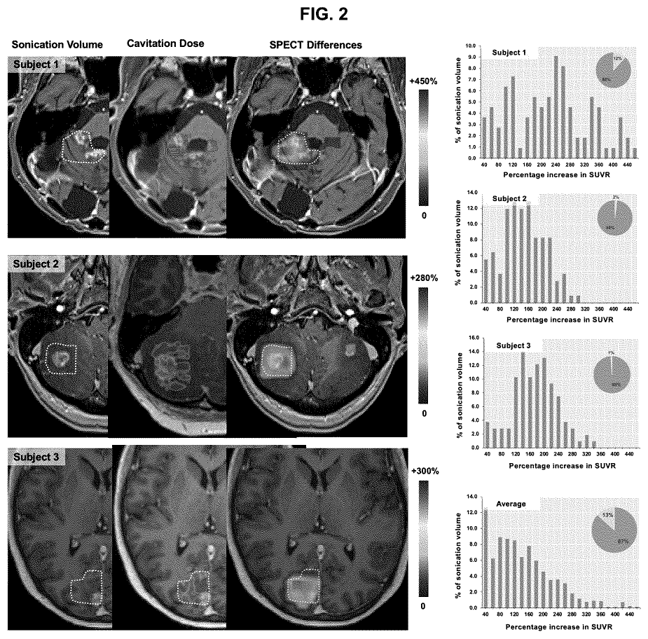

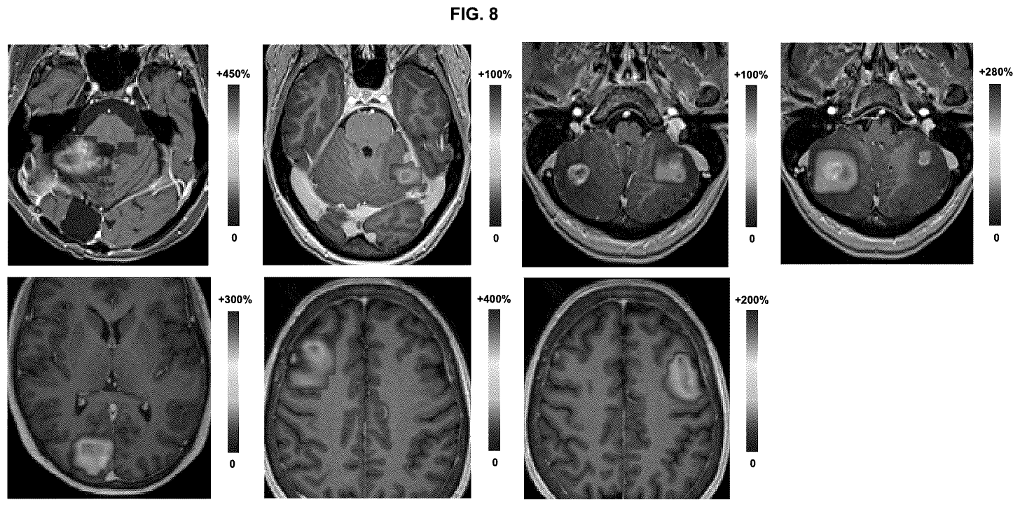

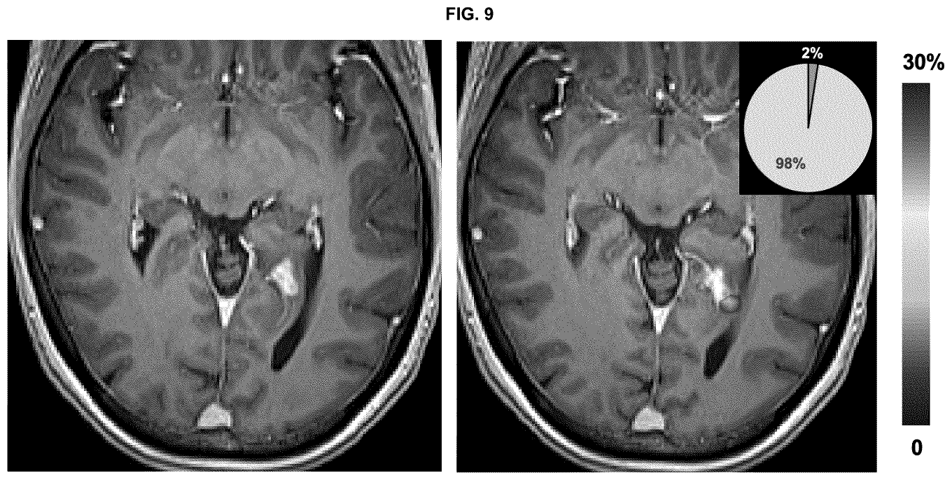

Finally, maps of the SUVR differences from the delayed imaging exquisitely correlate with the contour of sonication volumes (FIG. 2, FIG. 8). Voxel level analysis show delayed SUVR increased by as much as 450%, with on average 87% (SD 18%, range 49-99%) of sonication volume voxels with greater than 20% increase in SUVR, compared to 8% (SD 8%, range 2-14%) in control lesions (FIG. 2, FIG. 9).

TABLE 5
Summary of SUVmax and SUVRs in sonicated lesions

| | | Early 4 hrs SPECT imaging | | | Late 48 hrs SPECT imaging | | |
|---|---|---|---|---|---|---|---|
| | | Pre | Post | | Pre | Post | |
| | | FUS | FUS | Change | FUS | FUS | Change |
| Subject | Lesion | SUVR | SUVR | (%) | SUVR | SUVR | (%) |
| 1 | 1 | 1.18 | 2.20 | +86% | 1.84 | 4.32 | +135% |
| 2 | 1 | 1.64 | 2.55 | +55% | 3.80 | 4.26 | +12% |
| | 2 | 1.48 | 3.04 | +105% | 4.12 | 5.38 | +6% |
| | 3 | 1.69 | 2.16 | +28% | 2.60 | 5.64 | +117% |
| 3 | 1 | 0.82 | 1.17 | +43% | 1.20 | 3.42 | +184% |
| | 2 | 0.88 | 0.97 | +10% | 0.95 | 2.72 | +187% |
| | 3 | 1.47 | 2.19 | +49% | 1.94 | 5.03 | +160% |
| | Mean (SD) | 1.31 | 2.04 | | 2.35 | 4.40 | |
| | | (0.35) | (0.73) | | (1.22) | (1.06) | |

TABLE 6
Summary of SUVmax and SUVRs in unsonicated lesions

| | Early 4 hrs SPECT imaging | | | Late 48 hrs SPECT imaging | | |
|---|---|---|---|---|---|---|
| | | Post | | | Post | |
| | Pre FUS | FUS | Change | Pre FUS | FUS | Change |
| Lesion | SUVR | SUVR | (%) | SUVR | SUVR | (%) |
| 1 | 1.73 | 2.15 | +24% | 4.34 | 2.75 | −37% |
| 2 | 2.96 | 2.73 | −7% | 3.24 | 3.25 | 0% |
| Mean (SD) | 2.34 | 2.44 | | 3.79 | 3.00 | |
| | (0.87) | (0.41) | | (0.78) | (0.35) | |

### Example 7: Radiographic Response

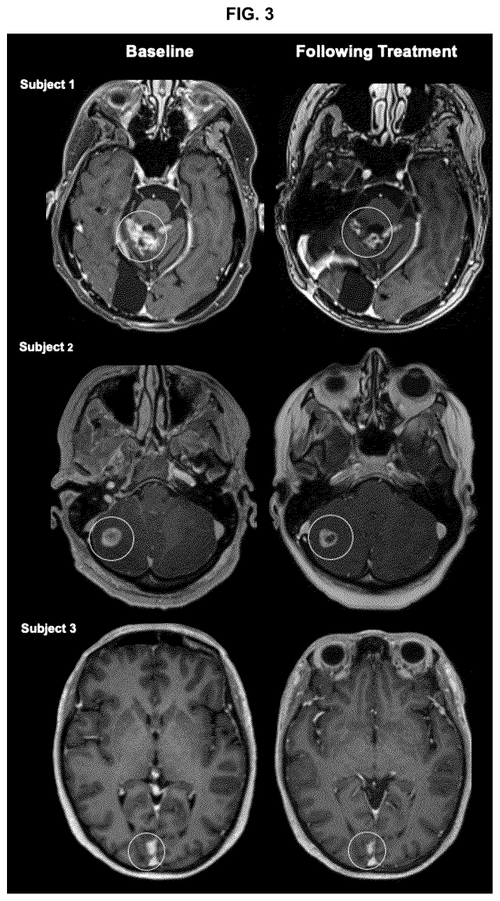

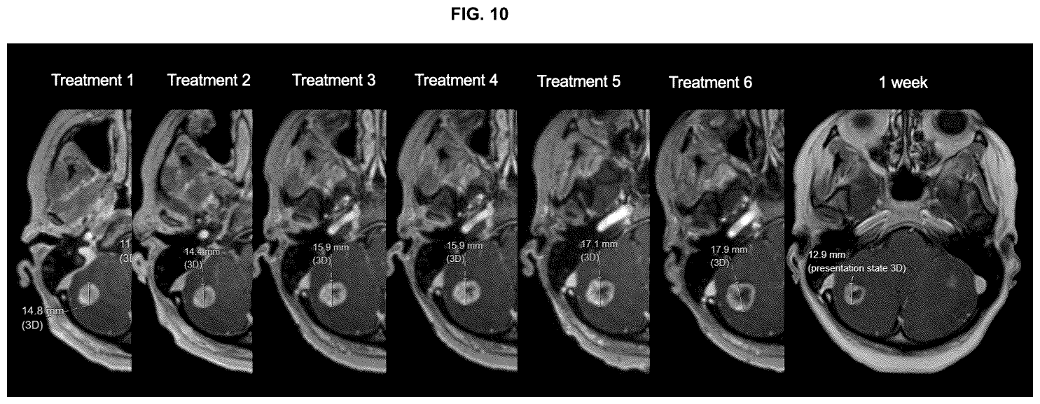

The experiments of this example show how all target tumors were either stable or reduced in size on the last follow-up MRI relative to baseline (FIG. 3). The dimensions are presented in Table 7, below. The sum of the longest diameters was reduced by 21% (SD 10%), and specifically by 25%, 7%, and 31% in subjects one, two, and three respectively. The reduction in enhancement and peri-tumor edema in subject one persisted at one year from initial treatment, while growth was seen in a region outside the sonication field. In subject two, a progressive growth was observed with central necrosis at the right cerebellar lesion over the course of treatment, followed by a significant size reduction, from maximum diameter of 18 to 13 mm (FIG. 10).

TABLE 7
Sum of the longest diameters, and sum of bidimensional
measurements (product of the longest diameter

| | Unidimensional | | | Bidimensional | | |
|---|---|---|---|---|---|---|
| | | Post- | | | Post- | |
| | Baseline | treatment | Change | Baseline | treatment | Change |
| Subject | (mm) | (mm) | (%) | (mm²) | (mm²) | (%) |
| 1 | 33.3 | 25.0 | −25 | 513 | 295 | −42 |
| 2 | 63.2 | 58.9 | −7 | 702 | 640 | −9 |
| 3 | 28.8 | 18.9 | −31 | 214 | 95 | −56 |

The experiments described in the examples above demonstrate the unexpected non-invasive, spatially targeted enhanced delivery of trastuzumab to a metastatic brain tumor using focused ultrasound. Using a novel radiopharmaceutical analogue, significantly increased radiotracer uptake was visualized compared to baseline, confirming target engagement across the permeabilized BBB. This was achieved through a safe, and transient opening in the BBB using transcranial MRgFUS, with reconstitution of the BBB within 24 hours of the procedure. The experiments described in the examples above demonstrate that MRgFUS can be feasibly combined with an existing regimen of targeted therapy, with resultant radiographic tumor response.

The procedures, in total fifteen, were well-tolerated with two cases of mild and transient AEs, and with all subjects discharged on the same day. Successful BBB opening using acoustic emissions during sonications was achieved. Overall, there is a observable efficacy signal of the treatments in the brain.

While subject one had confirmed Her2-positive brain metastases on pathology, the uptake and retention of radiolabeled trastuzumab in the other lesions is indicative of Her2 positivity.

Furthermore, eligible patients must be at least six weeks after any radiation treatment, and therefore the anti-tumor efficacy is likely due to treatment effect.

In summary, the present disclosure is the first direct evidence of non-invasive, image-guided, delivery of antibody therapy across the BBB, demonstrating improved therapeutic binding and activity in patients with progressive Her2-positive brain metastases.

### Methods

This was a prospective phase I, open-label, single-arm clinical trial, with an objective to demonstrate safety and feasibility of brain delivery of trastuzumab using MRgFUS. Eligible patients included those between 18 and 80 years of age with Her2-positive breast cancer and metastasis to the brain that are clearly defined on contrast enhanced MRI and progression on imaging. Eligibility criteria are described in Table 7, below. Three patients with Her2-positive breast cancer, progressive intracranial disease and stable systemic disease were enrolled. The treatments combined MRgFUS targeting of brain lesions with concomitant trastuzumab-based therapies. Indium-111 (¹¹¹In)-labelled trastuzumab was used to visualize drug biodistribution on SPECT imaging.

TABLE 7
Inclusion and exclusion criteria

Inclusion
Men or women between 18 and 80 years
Metastatic Her2-positive breast cancer with brain metastases, that are
clearly defined on pre-therapy contrast enhanced MRI, that have
progressed based on imaging
Standard treatment with radiation and/or surgery is allowed after the first
week of evaluation
At least 14 days after last brain surgery
At least 6 weeks after last radiation treatment
Any previous systemic treatment is allowed, but 1-3 weeks should have
passed since the last dose
Karnofsky rating 70 to 100
ASA score 1 to 3
Exclusion
Increased intracranial pressure e.g. symptoms of lethargy and papilledema,
midline shift more than 10 mm
Evidence of recent intracranial hemorrhage within the last 2 weeks
Cerebral infarction within the past 12 months, TIA in the last 1 month
Factors preventing transcranial delivery of ultrasound, e.g. significant
calcification in the beam path such that system tools cannot tailor the
treatment, more than 30% of skull area traversed by beam path covered by
scars or lesions, clips or other metallic implanted objects in the skull or
brain except shunts
Unstable or ongoing cardiac disease including low left ventricular ejection
fraction, QT prolongation, or pulmonary disease, or renal dysfunction
Severe uncontrolled hypertension
Anticoagulation therapy or history of bleeding disorder or abnormal
coagulation labs
Cerebral or systemic vasculopathy
Insulin-dependent diabetes mellitus
Contraindication to MRI, MRI contrast, or ultrasound contrast agent
Pregnancy

In the study, participants underwent six treatments of MRgFUS plus trastuzumab-based therapy, along the standard dosing regimen. Follow-up visits and MRIs were scheduled at one and three months after the last treatment.

One outcome was the characterization of treatment-related adverse events (AEs), through clinical neurologic exams and neuroimaging studies. A second outcome was the feasibility of inducing BBB permeability changes in intracranial metastatic disease as measured by contrast enhanced T1-weighted MRI. The effect of MRgFUS BBB opening on pharmacokinetics of trastuzumab was measured through SPECT imaging with ¹¹¹In-BzDTPA-NLS-trastuzumab.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of treating a Her2+ metastatic breast tumor in the brain, comprising:
(a) selecting a human patient having a Her2+ metastatic breast tumor in the brain or a recent resection of a Her2+ metastatic breast tumor in the brain; and
(b) applying an ultrasound beam to the cranium of the human patient to cause transient disruption of the blood-brain barrier (BBB) of the selected human patient,
wherein, the selected human patient is receiving:
one or more antibodies targeting Her2 during and/or after the application of the ultrasound beam, and
one or more microbubble compositions immediately before and/or during the application of the ultrasound beam.

2. The method of claim 1, wherein the ultrasound beam is a focused beam and/or wherein the ultrasound beam is a guided ultrasound beam.

3. The method of claim 1 or 2, wherein the ultrasound beam is a magnetic resonance-guided ultrasound beam, a computerized tomography (CT)-guided ultrasound beam, a positron emission tomography (PET)-guided ultrasound beam, or a stereotactically-navigated guided ultrasound beam, optionally a focused, magnetic resonance-guided ultrasound beam (MRgFUS).

4. The method of claim 3, wherein the application of the ultrasound beam targets at least one, or two, or three regions or sites of the brain.

5. The method of any one of claims 1-4, wherein the application of the ultrasound beam targets at least one, or two, or three regions or sites of the brain contemporaneously.

6. The method of any one of claims 1-5, wherein the treatment method is repeated at least two, or three, or four, or five, or ten times.

7. The method of any one of claims 1-6, wherein the selected human patient presents with a plurality of lesions.

8. The method of claim 7, wherein the selected human patient presents with a plurality of lesions in at least two regions or sites of the brain.

9. The method of any one of claims 1-6, wherein the selected human patient has had recent resection of the tumor and presents with a plurality of post-resection cavities.

10. The method of claim 9, wherein the selected human patient has had recent resection the tumor and presents with a plurality of post-resection cavities in at least two regions or sites of the brain.

11. The method of any one of claims 1-10, wherein the focused ultrasound beam is applied directly to the human patient's cranium using a helmet-shaped ultrasound transducer.

12. The method of any one of claims 1-11, wherein the focused ultrasound beam is applied at a center frequency of about 220 KHz.

13. The method of any one of claims 1-12, wherein the treatment duration is at least about 60 minutes, or at least about 90 minutes, or at least about 120 minutes, or at least about 150 minutes, or at least about 180 minutes.

14. The method of any one of claims 1-12, wherein the treatment duration is about 100 minutes, or about 110 minutes, or about 120 minutes, or about 130 minutes, or about 140 minutes, or about 150 minutes, or about 160 minutes.

15. The method of any one of claims 1-14, wherein the focused ultrasound beam is applied for at least about 10 seconds, or at least about 20 seconds, or at least about 30 seconds, or at least about 40 seconds, or at least about 50 seconds, or at least about 60 seconds.

16. The method of any one of claims 1-14, wherein the focused ultrasound beam is applied for about 10 seconds, or about 20 seconds, or about 30 seconds, or about 40 seconds, or about 50 seconds, or about 60 seconds.

17. The method of any one of claims 1-16, wherein the focused ultrasound beam is applied in pulses.

18. The method of any one of claims 1-17, wherein the focused ultrasound beam is applied at a power of at least about 5 W, or at least about 10 W, or at least about 15 W, or at least about 20 W, or at least about 25 W.

19. The method of any one of claims 1-17, wherein the focused ultrasound beam is applied at a power of about 10 W, or about 15 W, or about 20 W.

20. The method of any one of claims 1-19, wherein the focused ultrasound beam targets and/or the tumor is present in or has been resected from one or more of the frontal lobe, parietal lobe, temporal lobe, occipital lobe, and cerebellum.

21. The method of any one of claims 1-19, wherein the focused ultrasound beam targets and/or the tumor is present in or has been resected from the supratentorial region of the brain.

22. The method of any one of claims 1-19, wherein the focused ultrasound beam targets and/or the tumor is present in or has been resected from the infratentorial region of the brain.

23. The method of any one of claims 1-19, wherein the focused ultrasound beam targets and/or the tumor is present in or has been resected from the insula.

24. The method of any one of claims 1-19, wherein the focused ultrasound beam targets and/or the tumor is present in or has been resected from the brainstem.

25. The method of any one of claims 1-19, wherein the focused ultrasound beam targets and/or the tumor is present in or has been resected from the pons.

26. The method of any one of claims 1-19, wherein the focused ultrasound beam targets and/or the tumor is present in or has been resected from the posterior fossa.

27. The method of any one of claims 1-19, wherein the focused ultrasound beam targets the and/or the tumor is present in or has been resected from corticomedullary gray/white junction.

28. The method of any one of claims 1-19, wherein the focused ultrasound beam targets the and/or the tumor is present in or has been resected from meninges.

29. The method of any one of claims 1-28, wherein the antibody targeting Her2 is a full length antibody or an antibody format.

30. The method of claim 29, wherein the antibody format is selected from a single-chain antibody (scFv), a microprotein (cysteine knot protein, knottin), a DARPin; a Tetranectin; an Affibody; a Transbody; an Anticalin; an AdNectin; an Affilin; a Microbody; a plastic antibody; a single-domain antibody, a recombinant heavy-chain-only antibody (VHH), a shark heavy-chain-only antibody (VNAR), a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; an Affimer, a DuoBody, a Fv, a Fab, a Fab′, and a F(ab′)₂.

31. The method of any one of claim 29 or 30, wherein the antibody targeting Her2 is selected from pertuzumab, trastuzumab, and ado-trastuzumab.

32. The method of claim 31, wherein the antibody targeting Her2 is trastuzumab.

33. The method of claim 32, wherein the trastuzumab is administered at a dose of about 6 mg/kg to about 8 mg/kg.

34. The method of any one of claims 1-33, wherein the treatment further comprises administering an antibody-based anti-tumor combination agent.

35. The method of claim 34, wherein the antibody-based anti-tumor combination agent is a full length antibody or an antibody format.

36. The method of claim 35, wherein the antibody format is selected from a single-chain antibody (scFv), a microprotein (cysteine knot protein, knottin), a DARPin; a Tetranectin; an Affibody; a Transbody; an Anticalin; an AdNectin; an Affilin; a Microbody; a plastic antibody; a single-domain antibody, a recombinant heavy-chain-only antibody (VHH), a shark heavy-chain-only antibody (VNAR), a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; an Affimer, a DuoBody, a Fv, a Fab, a Fab′, and a F(ab′)₂.

37. The method of claim 35, wherein the antibody-based anti-tumor combination agent is a monoclonal antibody.

38. The method of claim 35, wherein the antibody-based combination agent is a bispecific antibody.

39. The method of any one of claims 34-38, wherein the antibody-based anti-tumor combination agent is an antibody-drug conjugate.

40. The method of any one of claims 34-39, wherein the antibody-based anti-tumor combination agent is directed to an antigen expressed on a tumor cell or an immune cell.

41. The method of any one of claims 34-40, wherein the antibody-based anti-tumor combination agent is directed to one of:
CD20, optionally selected from ibritumomab tiuxetan, obinutuzumab, ofatumumab, and rituximab;
CD30, optionally brentuximab;
CD52, optionally alemtuzumab;
EGFR, optionally selected from cetuximab, panitumumab, and necitumumab;
VEGF and VEGFR2, optionally selected from bevacizumab and ramucirumab;
programmed cell death protein 1 (PD-1), optionally selected from nivolumab, cemiplimab and pembrolizumab;
programmed cell death ligand 1 (PD-L1), optionally selected from atezolizumab, avelumab, and durvalumab;
CTLA-4, optionally ipilimumab; and
CD38, optionally daratumumab.

42. The method of any one of claims 34-41, wherein the tumor express the antigen against which the antibody-based anti-tumor combination agent is directed.

43. The method of any one of claims 1-42, wherein the antibody targeting Her2 is administered at least about 90 minutes, or at least about 120 minutes, or at least about 150 minutes, or at least about 180 minutes, or at least about 210 minutes before the application of the ultrasound beam.

44. The method of any one of claims 1-42, wherein the antibody targeting Her2 is administered about 60 minutes, or about 90 minutes, or about 120 minutes before the application of the ultrasound beam.

45. The method of any one of claims 1-42, wherein the antibody targeting Her2 is administered during the application of the ultrasound beam.

46. The method of any one of claims 1-42, wherein the antibody targeting Her2 is administered after the application of the ultrasound beam.

47. The method of any one of claims 1-46, wherein the antibody targeting Her2 is administered by systemic injection, bolus injection or slow diffusion injection.

48. The method of any one of claims 1-42, wherein the antibody targeting Her2 is administered by systemic infusion.

49. The method of any one of claims 1-48, wherein the microbubble compositions comprise one or more lipid-based microspheres.

50. The method of claim 49, wherein the microbubble compositions are perflutren lipid microspheres.

51. The method of any one of claims 1-50, wherein the microbubble compositions are administered to the patient no more than 60, or 30, or 20, or 10 minutes before the application of the ultrasound beam.

52. The method of any one of claims 1-51, wherein the microbubble compositions are administered to the patient throughout the method.

53. The method of any one of claims 1-52, wherein the microbubble compositions are administered by systemic injection, bolus injection or slow diffusion injection.

54. The method of claim 53, wherein the microbubble compositions are administered by systemic infusion.

55. The method of any one of claims 1-54, wherein substantially all of the disrupted BBB closes after the application of the ultrasound beam.

56. The method of any one of claims 1-55, wherein the transient disruption of the BBB allows for movement of the antibody targeting Her2 across the BBB.

57. The method of any one of claims 1-56, wherein the human patient demonstrates an increased standard uptake value ratio (SUVr) of greater than about 50%, or greater than about 60%, or greater than about 70%, or greater than about 80%, or greater than about 90%, as compared to administration of antibody targeting Her2 in the absence of application of the focused ultrasound beam to the cranium.

58. The method of any one of claims 1-56, wherein the human patient demonstrates an increased standard uptake value ratio (SUVr) of greater than about 50%, or greater than about 60%, or greater than about 70%, or greater than about 80%, or greater than about 90%, as compared to administration of the antibody targeting Her2 in the absence of application of the focused ultrasound beam to the cranium.

59. The method of any one of claims 1-58, wherein the tumors do not substantially increase in size after completion of the method.

60. The method of any one of claims 1-59, wherein the tumors reduce in size after completion of the method.

61. The method of any one of claims 1-60, wherein the tumors present as necrotic after completion of the method.

62. The method of claim 1, wherein the one or more antibodies targeting Her2 are labeled with a tracer label, to permit tracking of the transit of the treatment agent, wherein the tracer label optionally comprises indium-111.

63. The method of claim 62, wherein therapeutic delivery is quantified to determine the effect of improved therapeutic delivery across the BBB specific to the treatment agent being delivered

64. The method of claim 62 or 63, wherein the effect of ultrasound beam, optionally MRgFUS, BBB treatment on therapeutic delivery of the antibody targeting Her2 is measured by the imaging tracer signal, optionally on SPECT/CT images co-registered to clinical contrast-enhanced T1-weighted MRI to measure standardized uptake values (SUV) within the target tumor regions as measured within each tumor voxel.

65. The method of any one of claims 62-64, wherein the SUVs are normalized to an appropriate control region's mean SUV, optionally motor cortex or other normal brain, to calculate the standardized uptake value ratios (SUVRs) of each voxel across the entire tumor volume.

66. The method of any one of claims 62-65, wherein two-dimensional and/or three-dimensional volumetric heatmaps of a voxel-by-voxel percentage change in SUVR are generated using the formula:
(post-MRgFUS SUVR−baseline SUVR)/(baseline SUVR)
to characterize the effect of MRgFUS BBB treatment on improving the therapeutic delivery across the entire target region of the tumor volume.

67. A method for tracking therapeutic delivery and/or of an antibody-based treatment agent, comprising:
(a) selecting a human patient having a tumor in the brain or a recent resection of a tumor in the brain; and
(b) applying an ultrasound beam, optionally MRgFUS, to the cranium of the human patient to cause transient disruption of the BBB of the selected human patient;
wherein, the selected human patient is receiving:
one or more imaging tracer-labeled antibody-based treatment agents during and/or after the application of the ultrasound beam to allow tracking of the transit of the treatment agent, and
one or more microbubble compositions immediately before and/or during the application of the ultrasound beam.

68. The method of claim 67, wherein the method detects antibody-based treatment agent biodistribution in the body and/or across the BBB to a target brain region.

69. The method of claim 68, wherein the tracer is or comprises a radiolabel, optionally selected from one or more of indium-111 (¹¹¹In), fluorine-18 (¹⁸F), and carbon-11 (¹¹C).

70. The method of any one of claims 67-69, wherein therapeutic delivery to target brain regions via imaging tracer signal is quantified to determine an effect of therapeutic delivery across the BBB specific to the antibody-based treatment agent being delivered

71. The method of any one of claims 67-70, wherein an effect of ultrasound beam, optionally MRgFUS, treatment on the antibody-based treatment agent delivery is measured by the imaging tracer signal on SPECT/CT images co-registered to clinical contrast-enhanced T1-weighted MRI to measure standardized uptake values (SUV) within the target tumor regions as measured within each tumor voxel.

72. The method of claim 71, wherein the SUVs are normalized to an appropriate control region's mean SUV to calculate the standardized uptake value ratios (SUVRs) of each voxel across the entire tumor volume.

73. The method of claim 72, wherein the control region is selected from a motor cortex or other normal brain region.

74. The method of any one of claims 71-73, wherein two-dimensional and/or three-dimensional volumetric heatmaps of the voxel-by-voxel percentage change in SUVR are generated using the formula:
(post-MRgFUS SUVR−baseline SUVR)/(baseline SUVR)
to characterize the effect of MRgFUS BBB treatment on improving the therapeutic delivery across the entire target region of the tumor volume.

75. The method of any one of claims 67-74, wherein the tumor in the brain is metastatic.

76. The method of any one of claims 67-75, wherein the focused ultrasound beam is applied directly to the human patient's cranium using a helmet-shaped ultrasound transducer.

77. The method of any one of claims 67-76, wherein the focused ultrasound beam is applied at a center frequency of about 220 KHz.

78. The method of any one of claims 67-77, wherein the microbubble compositions comprise one or more lipid-based microspheres.

79. The method of claim 78, wherein the microbubble compositions are perflutren lipid microspheres.

80. The method of any one of claims 67-79, wherein the microbubble compositions are administered to the patient no more than 60, or 30, or 20, or 10 minutes before the application of the ultrasound beam.

81. The method of any one of claims 67-80, wherein the microbubble compositions are administered to the patient throughout the method.

82. The method of any one of claims 67-81, wherein the microbubble compositions are administered by systemic injection, bolus injection or slow diffusion injection.

83. The method of claim 82, wherein the microbubble compositions are administered by systemic infusion.

84. The method of any one of claims 67-83, wherein substantially all of the disrupted BBB closes after the application of the ultrasound beam.

85. The method of any one of claims 67-84, wherein the transient disruption of the BBB allows for movement of the antibody-based treatment agent across the BBB.

86. The method of any one of claims 67-85, wherein the human patient demonstrates an increased standard uptake value ratio (SUVr) of greater than about 50%, or greater than about 60%, or greater than about 70%, or greater than about 80%, or greater than about 90%, as compared to administration of antibody-based treatment agent in the absence of application of the focused ultrasound beam to the cranium.

87. The method of any one of claims 67-85, wherein the human patient demonstrates an increased standard uptake value ratio (SUVr) of greater than about 50%, or greater than about 60%, or greater than about 70%, or greater than about 80%, or greater than about 90%, as compared to administration of the antibody-based treatment agent in the absence of application of the focused ultrasound beam to the cranium.


SEQUENCE LISTING (1 sequence)

SEQ ID NO: 1 - protein, 13 aa, synthetic construct
CGYGPKKKRKVGG